(19) United States
(12) Patent Application Publication
(10) Pub. No.: US 2019/0314377 A1
(43) Pub. Date: Oct. 17, 2019

(54) TREATMENT OF HEMATOLOGICAL MALIGNANCY WITH SMALL MOLECULE NF-KB INHIBITORS

(71) Applicants: MEMORIAL SLOAN-KETTERING CANCER CENTER, New York, NY (US); IMMUNETARGET, INC., San Diego, CA (US)

(72) Inventors: Johannes ZAKRZEWSKI, New York, NY (US); Hsiou-Chi LIOU, New York, NY (US); Samedy OUK, San Diego, CA (US)

(21) Appl. No.: 16/453,424

(22) Filed: Jun. 26, 2019

Related U.S. Application Data
(63) Continuation of application No. PCT/US2017/068418, filed on Dec. 26, 2017.
(60) Provisional application No. 62/439,335, filed on Dec. 27, 2016.
(60) Provisional application No. 62/439,841, filed on Dec. 28, 2016.

(51) Int. Cl. A61K 31/519; A61K 31/505; A61K 31/4045; A61P 35/00

(52) U.S. Cl. CPC A61K 31/519; A61P 35/00; A61K 31/4045; A61K 31/505

(57) ABSTRACT

The disclosure relates to compounds and compositions that inhibit NF-κB and are useful in the treatment of hematological malignancies such as lymphoma and myeloma.

### CROSS REFERENCE TO RELATED APPLICATIONS

This application is a continuation of PCT/US2017/068418 filed Dec. 26, 2017 and published on Jul. 5, 2018 as WO 2018/125867, which claims priority to U.S. provisional application, 62/439,335 filed Dec. 27, 2016 and U.S. provisional application, 62/439,841 filed Dec. 28, 2016; the contents of which are hereby incorporated by reference.

### GOVERNMENT SUPPORT

This invention was made with government support under CA160659 awarded by the National Institutes of Health. The government has certain rights in the invention.

### TECHNICAL FIELD

The present disclosure provides methods for the treatment of hematological malignancies using compounds and pharmaceutical compositions capable of inhibiting NF-κB.

### BACKGROUND OF THE DISCLOSURE

NF-κB/Rel (nuclear factor kappa B) is a family of transcription factors that includes p50/p105 (NF-κB1), p52/p100 (NF-κB2), p65 (RelA), c-Rel, and RelB. These molecules can homo- or heterodimerize, and are generally sequestered in the cytoplasm by their inhibitors, IκBs. Upon activation, IκBs are degraded by the 26s proteasome and NF-κB dimers migrate into the nucleus to perform transcriptional activity.

NF-κB (p50/p65) and c-Rel are regulated by the canonical IKKα/β/γ kinase complex pathway, whereas RelB and p52 (NF-kB2) are regulated by an alternative pathway via the IKKα/NIK complex. Despite this similarity, each NF-κB family member is distinct with regard to tissue expression pattern, response to receptor signals, and target gene specificity. These differences are evident from the non-redundant phenotypes exhibited by individual NF-κB/Rel knockout mice. Therefore, therapeutics targeted to different NF-κB/Rel members are likely to have different biological effects and toxicity profiles.

Many receptors and stimuli can activate NF-κB/Rel, including TCR/BCR, TNF receptor superfamily (e.g. CD40, TNFR1, TNFR2, BAFF, APRIL, RANK), IL-1/TLR receptors, and Nod-like receptors, as well as activating oncogenes (e.g. Src, Ras, LMP-1, Tax, v-FLIP), reactive oxygen radicals, radiation, and chemotherapeutic agents. In response to these stimuli, NF-κB/Rel regulates the expression of cytokines, chemokines, and molecules that play a role in adhesion, the cell cycle, apoptosis, and angiogenesis. As such, NF-κB/Rel transcription factors are important therapeutic targets for many human disorders, including inflammation, autoimmune diseases, and cancer, and small molecule inhibitors of NF-κB/Rel may be useful as therapeutics for these disorders.

### SUMMARY OF THE DISCLOSURE

In one aspect, the present disclosure relates to methods of treating hematological malignancies using compounds and compositions capable of inhibiting NF-kB.

The present disclosure is based on the observation that compounds selected from the group consisting of:

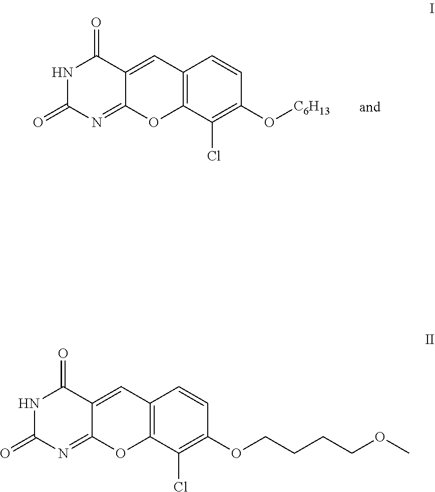

and crystalline forms, hydrates, or pharmaceutically acceptable salts thereof inhibit NF-κB.

In one aspect, therefore, the disclosure relates to a method for inhibiting growth and proliferation of leukemia cells, lymphoma cells, myeloma cells, chronic lymphocytic leukemia (CLL) cells, acute lymphocytic leukemia (ALL) cells, chronic myelogenous leukemia (CML) cells, acute myelogenous leukemia (AML) cells, diffuse large B-cell lymphoma (DLBCL) cells, multiple myeloma (MM) cells comprising contacting the cells with a compound selected from the group consisting of:

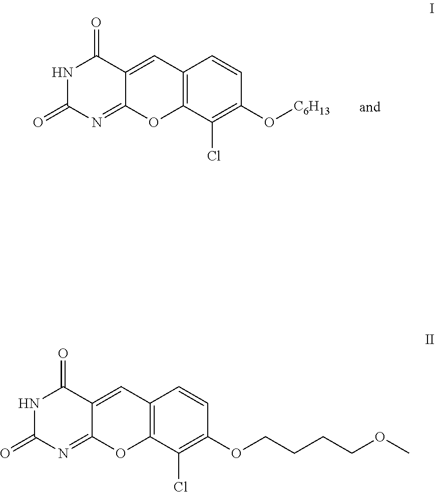

and crystalline forms, hydrates, or pharmaceutically acceptable salts thereof.

In one embodiment, the cells are lymphoma cells. In another embodiment, the cells are myeloma cells.

In one aspect, therefore, the disclosure relates to a method for inhibiting growth and proliferation of cells selected from the group consisting of lymphoma cells and myeloma cells comprising contacting the cells with a compound selected from the group consisting of:

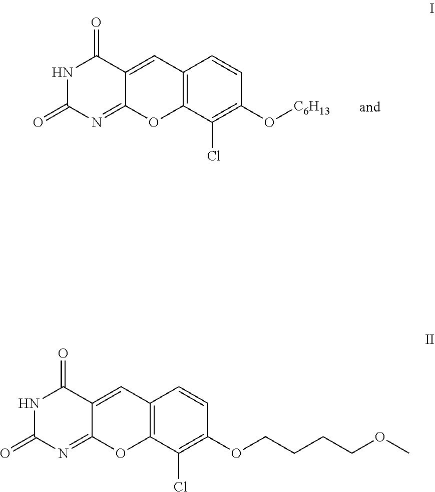

and crystalline forms, hydrates, or pharmaceutically acceptable salts thereof.

In yet another related aspect, the disclosure relates to a method for treating a subject with a malignancy selected from leukemia, lymphoma, myeloma, chronic lymphocytic leukemia (CLL), acute lymphocytic leukemia (ALL), chronic myelogenous leukemia (CML), acute myelogenous leukemia (AML), diffuse large B-cell lymphoma (DLBCL), and multiple myeloma (MM) by administering to the subject a therapeutically effective amount of a compound selected from the group consisting of:

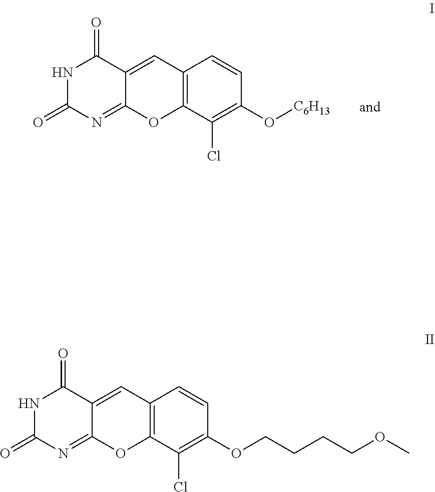

and crystalline forms, hydrates, or pharmaceutically acceptable salts thereof.

In yet another related aspect, therefore, the disclosure relates to a method for treating a subject with a lymphoma or myeloma by administering to the subject a therapeutically effective amount of a compound selected from the group consisting of:

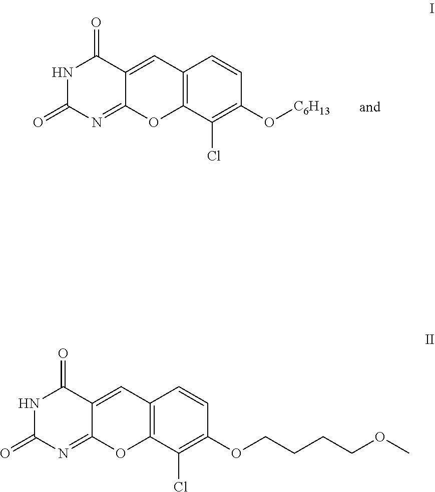

and crystalline forms, hydrates, or pharmaceutically acceptable salts thereof.

In one embodiment, the myeloma is multiple myeloma; in another embodiment, the lymphoma is diffuse large B-cell lymphoma (DLBCL).

In another related aspect, the disclosure relates to a method for treating a subject with a lymphoma or myeloma by administering to the subject a therapeutically effective amount of a compound selected from the group consisting of:

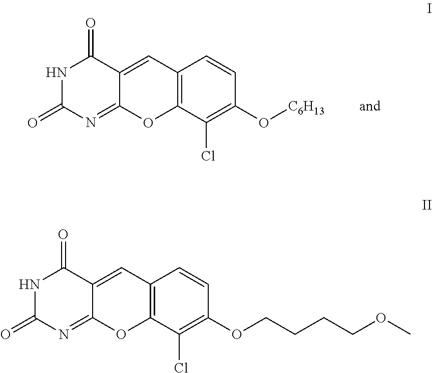

and crystalline forms, hydrates, or pharmaceutically acceptable salts thereof in combination with bortezomib or panobinostat.

In another related aspect, therefore, the present disclosure relates to a pharmaceutical composition comprising

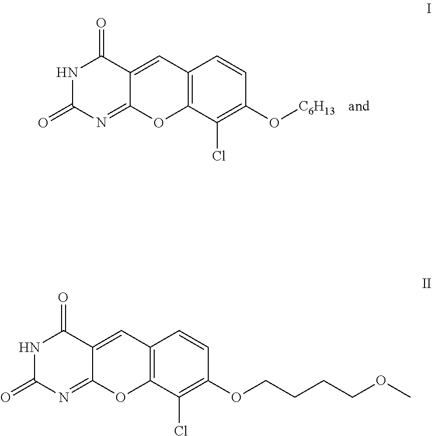

and crystalline forms, hydrates, or pharmaceutically acceptable salts thereof in combination with bortezomib or panobinostat.

### BRIEF DESCRIPTION OF THE DRAWINGS

FIG. 1 shows that IT-848 inhibits NF-κB activity in B cell lymphoma and multiple myeloma cells. Human diffuse large B cell lymphoma cells (HBL-1 and U2932) and multiple myeloma cells (MM.1S and U266) were cultured in the presence of IT-848 (2, 4 or 6 μM) or empty vehicle for 12 hours. NF-κB activity was analyzed by Western Blot of the NF-κB pathway signaling transduction molecules IKK-beta and IkB-alpha. Beta Actin was included as an internal control.

FIG. 2 shows that IT-848 is a more potent NF-κB inhibitor than IT-878. Jurkat/GFP/NF-κB transcriptional reporter cells were stimulated with TNF-α and incubated for 8 and 22 hours in the presence of empty vehicle or 1, 3 and 6 μM of IT-848 or IT-878. NF-κB transcriptional activity (GFP mean fluorescence intensity) was analyzed by flow cytometry. Mean and SEM of relative fluorescent intensities are presented.

FIG. 3 shows that IT-848 and IT-878 are specific inhibitors of NF-κB transcriptional activity. Jurkat/GFP/NF-κB transcriptional reporter cells were stimulated with PMA/ionomycin, and HepG2/Luciferase/Nrf2-Antioxidant response element (ARE) transcriptional reporter cells were treated with tert-butylhydroquinone. The respective cell lines were incubated for 12 hours in the presence of empty vehicle or 1, 3, 6 and 10 μM of IT-848 or IT-878. NF-κB transcriptional activity was analyzed by flow cytometry, NFAT and Nrf2 transcriptional activities were analyzed by luminescence measurement. Mean and SEM of relative fluorescent or luminescent intensities are presented.

FIG. 4 shows that treatment of healthy cells with IT-848 and IT-878 is associated with moderate toxicity. Mouse splenocytes and human peripheral blood mononuclear cells (PBMC) were incubated for 24 hours in the presence of empty vehicle or 1, 3, 6 and 10 μM of IT-848 or IT-878. Viability (percentage of DAPI-negative cells) was analyzed by flow cytometry and metabolic activity was analyzed by MTS assay. Mean and SEM of relative viability or metabolic activity are presented.

FIG. 5A shows that IT-848 inhibits growth of MM.1S multiple myeloma cells. MM.1S human multiple myeloma cells were incubated for 48 hours in the presence of empty vehicle or 2, 4 and 6 μM of IT-848, IT-878, PS-1145, or ibrutinib, Cell proliferation was analyzed after 24 and 48 hours by MTS assay. Mean and SEM or relative cell growth are presented.

FIG. 5B shows that IT-848 inhibits growth of U266 multiple myeloma cells. U266 human multiple myeloma cells were incubated for 48 hours in the presence of empty vehicle or 2, 4 and 6 μM of IT-848, IT-878, PS-1145, or ibrutinib, Cell proliferation was analyzed after 24 and 48 hours by MTS assay. Mean and SEM or relative cell growth are presented.

FIG. 5C shows that IT-848 inhibits growth of TMD8 B cell lymphoma cells. TMD8 human DLBCL cells were incubated for 48 hours in the presence of empty vehicle or 2, 4 and 6 μM of IT-848, IT-878, PS-1145, or ibrutinib, Cell proliferation was analyzed after 24 and 48 hours by MTS assay. Mean and SEM or relative cell growth are presented.

FIG. 5D shows that IT-848 inhibits growth of SUDHL4 B cell lymphoma cells. SUDHL4 human DLBCL cells were incubated for 48 hours in the presence of empty vehicle or 2, 4 and 6 μM of IT-848, IT-878, PS-1145, or ibrutinib, Cell proliferation was analyzed after 24 and 48 hours by MTS assay. Mean and SEM or relative cell growth are presented.

FIG. 6 shows that IT-848 modulates the redox state of multiple myeloma cells. MM.1S cells, U266 cells or mouse splenocytes were cultured in the presence of empty vehicle or serial dilutions of IT-848. ROS levels were quantified after 4 hours and 24 hours by DCFDA assay.

FIG. 7 shows the pharmacokinetics of IT-848. IT-848 is cleared slowly after systemic administration.

FIGS. 8A and 8B show that IT-848 enhances the efficacy of Bortezomib in a xenograft model of multiple myeloma. NSG mice received 2.5×10⁶ luciferase-expressing MM.1S cells intravenously. After 10 days engraftment was confirmed and mice were assigned to four groups: empty vehicle i.p. M/W/F×4 weeks; Bortezomib 0.5 mg/kg i.p. M/T×4 weeks; IT-848 10 mg/kg i.p. M/W/F×4 weeks; Bortezomib M/T+IT-848 M/W/F×4 weeks. A. Tumor growth was analyzed by in vivo bioluminescence imaging. B. Mean and SEM of bioluminescence intensities are presented. N=7-10 The area under the curve (AUC) was used to summarize the bioluminescence intensity for each subject and a permutation test was performed to determine if there was a difference in the AUCs between groups. P values are as follows: Group 1 vs. 2: 0.008; Group 2 vs. 3: 0.032; Group 1 vs 3: <0.001; Group 2 vs 4: <0.001; Group 1 vs 4: <0.001; Group 3 vs 4: 0.029.

FIG. 9 Mice were assigned to three treatment groups (IT-848 single agent, bortezomib single agent, combination of IT-848 and bortezomib) and a control group (empty vehicle). Onset of hind leg paralysis, which is the clinical endpoint of progressive disease in this model, was delayed in all three treatment groups (FIG. 9). In vivo BLI (FIG. 8A) including statistical analysis of longitudinal BLI data (FIG. 8B) revealed that both IT-848 and bortezomib were efficacious but combination treatment was best.

FIG. 10 shows IT-848 inhibits expression of the Nf-kB target gene interleukin 6 (IL-6). TMD8, HBL1 and U266 cells were incubated for 24 hours in the presence of empty vehicle or IT-848 (4 and 6 μM). IL-6 concentrations in the culture media were analyzed after 12 and 24 hours by ELISA. Mean and SEM of relative IL-6 levels (percentage of vehicle) are presented.

FIG. 11 shows that IT-848 inhibits expression of the Nf-kB target gene interleukin 10 (IL-10). TMD8, HBL1 and U266 cells were incubated for 24 hours in the presence of empty vehicle or IT-848 (4 and 6 μM). IL-10 concentrations in the culture media were analyzed after 12 and 24 hours by ELISA. Mean and SEM of relative IL-10 levels (percentage of vehicle) are presented.

FIG. 12 shows that IT-848 enhances the activity of the histone deacetylase inhibitor Panobinostat against TMD8 cells. TMD8 and DLBCL cells were incubated for 48 hours in the presence of empty vehicle, IT-848 (4 μM), Panobinostat (0.1 μM), or a combination of IT-848 and Panobinaostat. Cell proliferation was analyzed after 24 and 48 hours by MTS assay. Mean and SEM of relative cell growth are presented.

FIG. 13 shows that IT-848 enhances the activity of the proteasome inhibitor Bortezomib against HBL1 cells. HBL1 human DLBCL cells were incubated for 48 hours in the presence of empty vehicle, IT-848 (4 μM), Bortezomib (0.02 μM), or a combination of IT-848 and Bortezomib. Cell proliferation was analyzed after 24 and 48 hours by MTS assay. Mean and SEM of relative cell growth are presented.

### DETAILED DESCRIPTION OF THE DISCLOSURE

All patents, published applications and other publications and references are hereby incorporated by reference into the present disclosure in their entirety.

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The term “about” generally indicates within ±0.5%, 1%, 2%, 5%, or up to ±10% of the indicated value. For example, an amount of “about 10 wt %” generally indicates, in its broadest sense, 10 wt %±10%, which indicates 9.0-11.0 wt %. The term “about” may alternatively indicate a variation or average in a physical characteristic of a group.

Compounds useful in practicing the disclosed methods may include the following: a compound selected from the group consisting of:

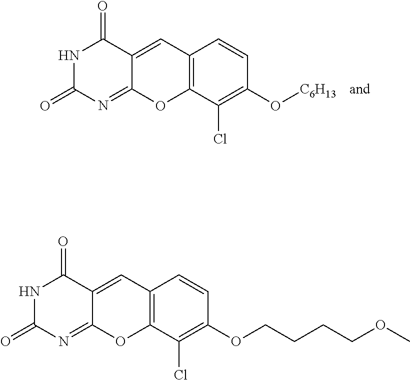

crystalline forms, hydrates, or pharmaceutically acceptable salts thereof.

As used herein, the term “compound” refers to two or more atoms that are connected by one or more chemical bonds. In the present disclosure, “chemical bonds” and “bonds” are interchangeable and include, but are not limited to, covalent bonds, ionic bonds, hydrogen bonds, and van der Waals interactions. Covalent bonds of the present disclosure include single, double, and triple bonds. Compounds of the present disclosure include, but are not limited to, organic molecules. Atoms that comprise the compounds of the present disclosure are “linked” if they are connected by a chemical bond of the present disclosure.

Organic compounds of the present disclosure include linear, branched, and cyclic hydrocarbons with or without functional groups. The term “C_{x-y}” when used in conjunction with a chemical moiety, such as, alkyl, alkenyl, alkynyl or alkoxy is meant to include groups that contain from x to y carbons in the chain. For example, the term “C_{x-y} alkyl” means substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. The terms “C_{x-y} alkenyl” and “C_{x-y} alkynyl” refer to substituted or unsubstituted unsaturated aliphatic groups analogous in length and possible substitution to the alkyls described above, but containing at least one double or triple bond respectively.

The term “independently selected” and grammatical variations thereof mean that, in a chemical structure of the present disclosure, (e.g., formula I), if more than one atom in the structure can be selected from a list of elements, those atoms may or may not be of the same element. Similarly, if more than one chemical moiety in the structure can be selected from a list of chemical moieties, those moieties may or may not be the same.

In the present disclosure, the compound capable of inhibiting NF-kB may function as a direct or indirect NF-kB inhibitor. A direct NF-kB inhibitor is a compound that binds to or interacts with NF-kB family members directly and inhibits its DNA binding and transcriptional function. An indirect NF-kB inhibitor is a compound that binds to or interacts with a compound other than NF-kB family members, thereby generating a downstream inhibitory effect on NF-kB activity.

As used herein, the term “contacting” means bringing a compound of the present disclosure into close proximity to the cells of the present disclosure. This may be accomplished using conventional techniques of drug delivery to mammals including but not limited to tail vein injection, intravenous injection, per oral or by addition of the compound to a culture media in which the cells of the present disclosure are located.

A further embodiment of the present disclosure is a method of inhibiting lymphoma or myeloma cells with any of the compounds disclosed herein and/or a pharmaceutically acceptable formulation thereof.

In the present disclosure, the term “crystalline form” means the crystal structure of a compound. A compound may exist in one or more crystalline forms, which may have different structural, physical, pharmacological, or chemical characteristics. Different crystalline forms may be obtained using variations in nucleation, growth kinetics, agglomeration, and breakage.

Nucleation results when the phase-transition energy barrier is overcome, thereby allowing a particle to form from a supersaturated solution. Crystal growth is the enlargement of crystal particles caused by deposition of the chemical compound on an existing surface of the crystal. The relative rate of nucleation and growth determine the size distribution of the crystals that are formed. The thermodynamic driving force for both nucleation and growth is supersaturation, which is defined as the deviation from thermodynamic equilibrium. Agglomeration is the formation of larger particles through two or more particles (e.g., crystals) sticking together and forming a larger crystalline structure.

The term “hydrate”, as used herein, means a solid or a semi-solid form of a chemical compound containing water in a molecular complex. The water is generally in a stoichiometric amount with respect to the chemical compound.

As used herein, “pharmaceutically acceptable salts” refer to derivatives of the compounds disclosed herein wherein the compounds are modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include salts from ammonia, L-arginine, betaine, benethamine, benzathine, calcium hydroxide, choline, deanol, diethanolamine (2,2′-iminobis(ethanol)), diethylamine, 2-(diethylamino)-ethanol, 2-aminoethanol, ethylenediamine, N-ethyl-glucamine, hydrabamine, 1H-imidazole, lysine, magnesium hydroxide, 4-(2-hydroxyethyl)-morpholine, piperazine, potassium hydroxide, 1-(2-hydroxy-ethyl)-pyrrolidine, sodium hydroxide, triethanolamine (2,2′,2″-nitrilotris(ethanol)), trometh-amine, zinc hydroxide, acetic acid, 2.2-dichloro-acetic acid, adipic acid, alginic acid, ascorbic acid, L-aspartic acid, benzenesulfonic acid, benzoic acid, 2,5-dihydroxybenzoic acid, 4-acetamido-benzoic acid, (+)-camphoric acid, (+)-camphor-10-sulfonic acid, carbonic acid, cinnamic acid, citric acid, cyclamic acid, decanoic acid, dodecylsulfuric acid, ethane-1,2-disulfonic acid, ethanesulfonic acid, 2-hydroxy-ethanesulfonic acid, ethylenediamonotetraacetic acid, formic acid, fumaric acid, galacaric acid, gentisic acid, D-glucoheptonic acid, D-gluconic acid, D-glucuronic acid, glutamic acid, glutantic acid, glutaric acid, 2-oxo-glutaric acid, glycero-phosphoric acid, glycine, glycolic acid, hexanoic acid, hippuric acid, hydrobromic acid, hydrochloric acid isobutyric acid, DL-lactic acid, lactobionic acid, lauric acid, lysine, maleic acid, (−)-L-malic acid, malonic acid, DL-mandelic acid, methanesulfonic acid, galactaric acid, naphthalene-1,5-disulfonic acid, naphthalene-2-sulfonic acid, 1-hydroxy-2-naphthoic acid, nicotinic acid, nitric acid, octanoic acid, oleic acid, orotic acid, oxalic acid, palmitic acid, pamoic acid (embonic acid), phosphoric acid, propionic acid, (−)-L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid. Further pharmaceutically acceptable salts can be formed with cations from metals like aluminum, calcium, lithium, magnesium, potassium, sodium, zinc and the like. (Pharmaceutical salts, Berge, S. M. et al., J. Pharm. Sci., (1977), 66, 1-19).

The pharmaceutically acceptable salts of the present disclosure can be synthesized from a compound disclosed herein which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

The compositions of the disclosure comprise one or more active ingredients in admixture with one or more pharmaceutically acceptable diluents or carriers and, optionally, one or more other compounds, drugs, ingredients and/or materials. Regardless of the route of administration selected, the agents/compounds of the present disclosure are formulated into pharmaceutically-acceptable dosage forms by conventional methods known to those of skill in the art. See, e.g., Remington, The Science and Practice of Pharmacy (21st Edition, Lippincott Williams and Wilkins, Philadelphia, Pa.).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the disclosure must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The compositions of the disclosure may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols; and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins; cocoa butter, starches, tragacanth, cellulose derivatives, polyethylene glycol, silicones, bentonites, silicic acid, talc, salicylate, zinc oxide, aluminum hydroxide, calcium silicates, and polyimide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol; oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient; such as sugars and sodium chloride; (26) thickening agents; (27) coating materials, such as lecithin; and (28) sweetening, flavoring; coloring; perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The compositions of the present disclosure suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The compositions of the present disclosure for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present disclosure which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The compositions of the present disclosure suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

Any formulation of the disclosure may be presented in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

### Use as Inhibitors of NF-κB Transcriptional Activity

Oxidative stress and NF-κB play differential roles in healthy and diseased cells. Manipulation of the cellular redox balance has the potential to be therapeutically exploitable in the setting of cancer. Constitutive activity of NF-κB family members such as c-Rel has been associated with oncogenesis and resistance to chemotherapy, making NF-κB a reasonable therapeutic target in a wide range of cancers including hematologic malignancies and solid tumors. There are multiple interactions between reactive oxygen and nitrogen species (ROS/RNS) and the NF-κB pathway: NF-κB is an important part of the oxidative stress response, while at the same time ROS are known to be able to inhibit NF-κB activity through thiol modifications of DNA binding domains. In this disclosure, compounds combining inhibition of NF-κB DNA binding with the capacity to modulate the redox balance of cancer cells, resulting in potent immunomodulatory properties and antitumor activity are described.

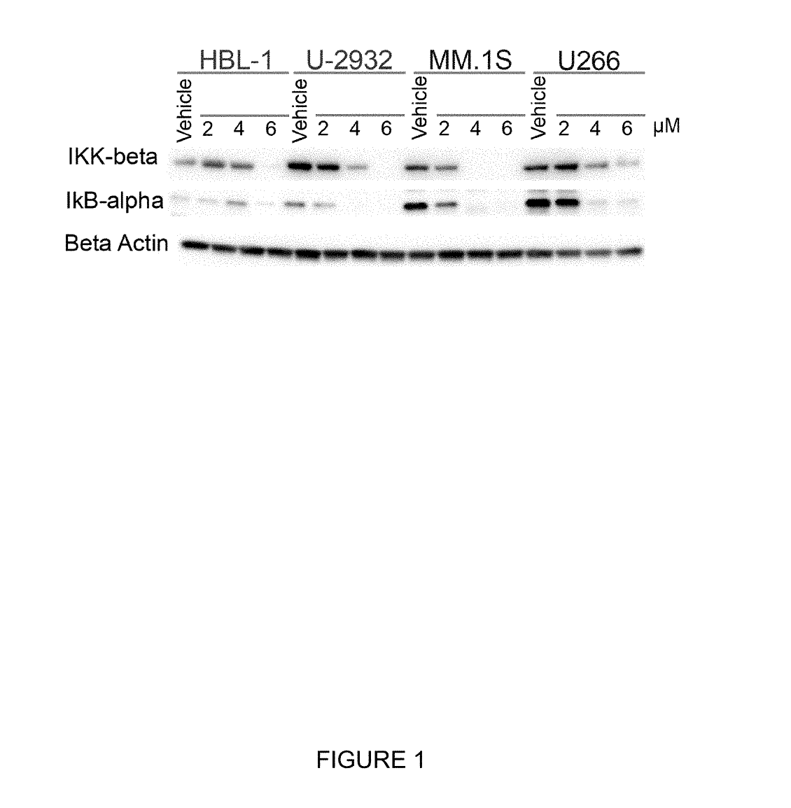

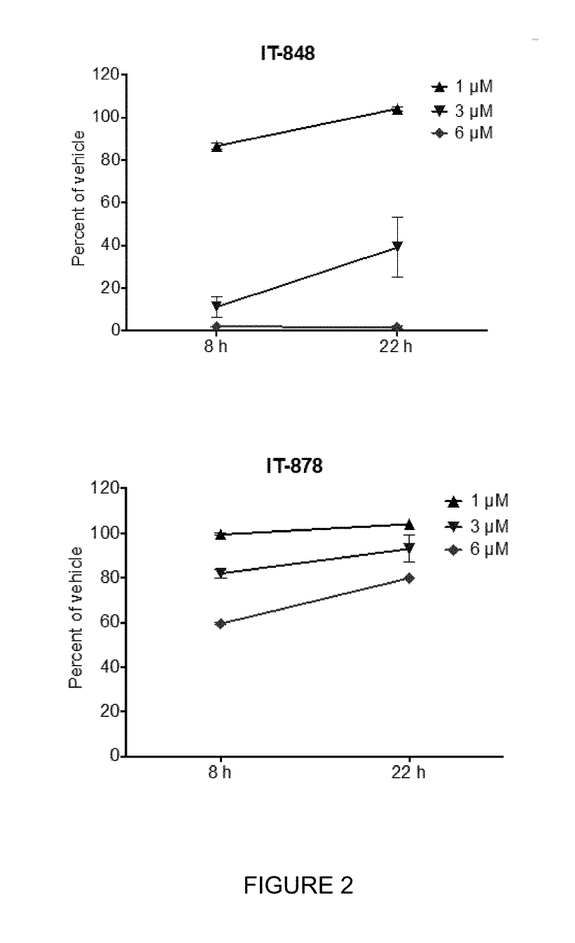

FIG. 1 shows a Western Blot confirming dose-dependent inhibition of NF-kB pathway activity by a compound of the disclosure, IT-848, in human B cell lymphoma and multiple myeloma cells. Using a NF-kB-GFP reporter cell line inhibition of NF-kB activity of two molecules, IT-848 and IT-878 was examined after eight and 22 hours of incubation (FIG. 2). IT-848 was more potent than IT-878 and resulted in complete inhibition of NF-kB activity at a concentration of 6 micromolar.

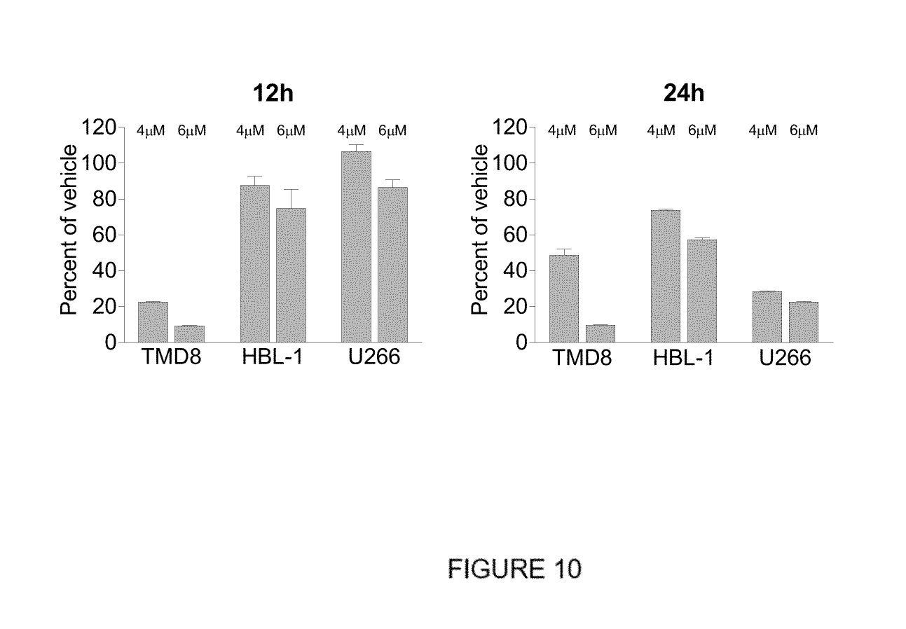

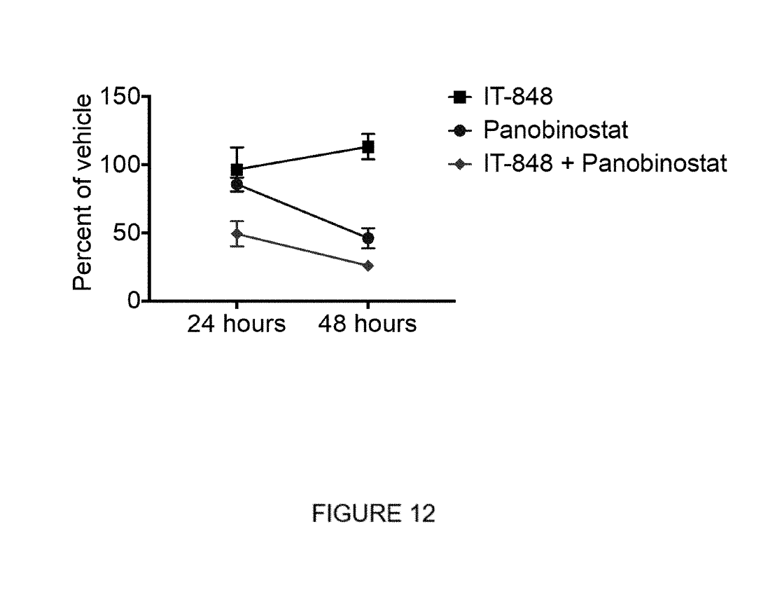

To confirm the potency of IT-848 we analyzed expression of surrogate markers of NF-kB activity in B cell malignancies (the NF-kB target genes IL-6 and IL-10) in diffuse large B cell lymphoma (DLBCL) and Multiple Myeloma (MM) cell lines treated with IT-848 (FIGS. 10+11). We found that secretion of both cytokines was decreased after 12 and 24 hours of treatment in TMD8 cells (DLBCL), HBL1 cells (DLBCL), and U266 cells (MM). We also performed efficacy studies of IT-848 combined with drugs that are currently used clinically to treat B cell malignancies. MTS assays in TMD8 and HBL1 cells revealed that IT-848 can enhance the efficacy of bortezomib or panobinostat (FIGS. 12+13)

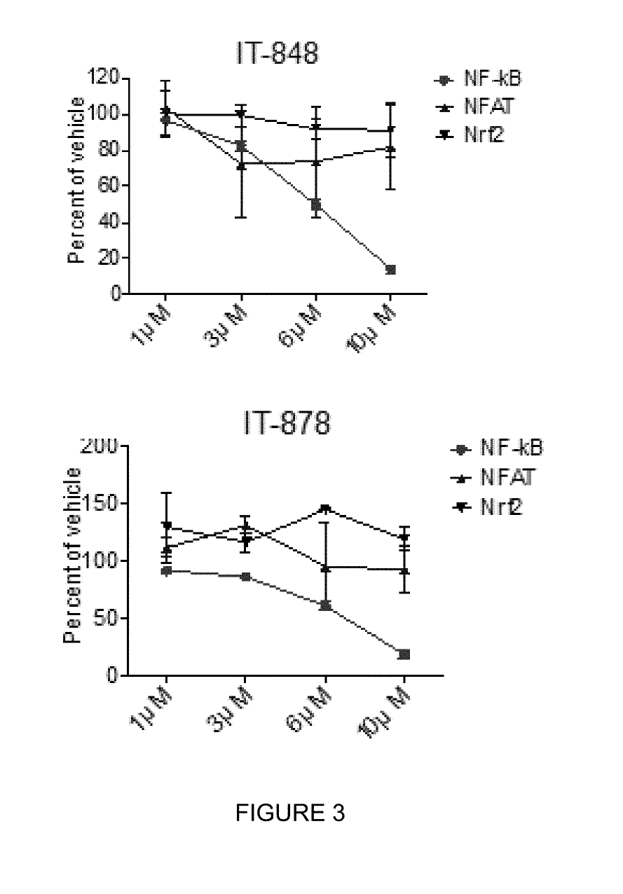

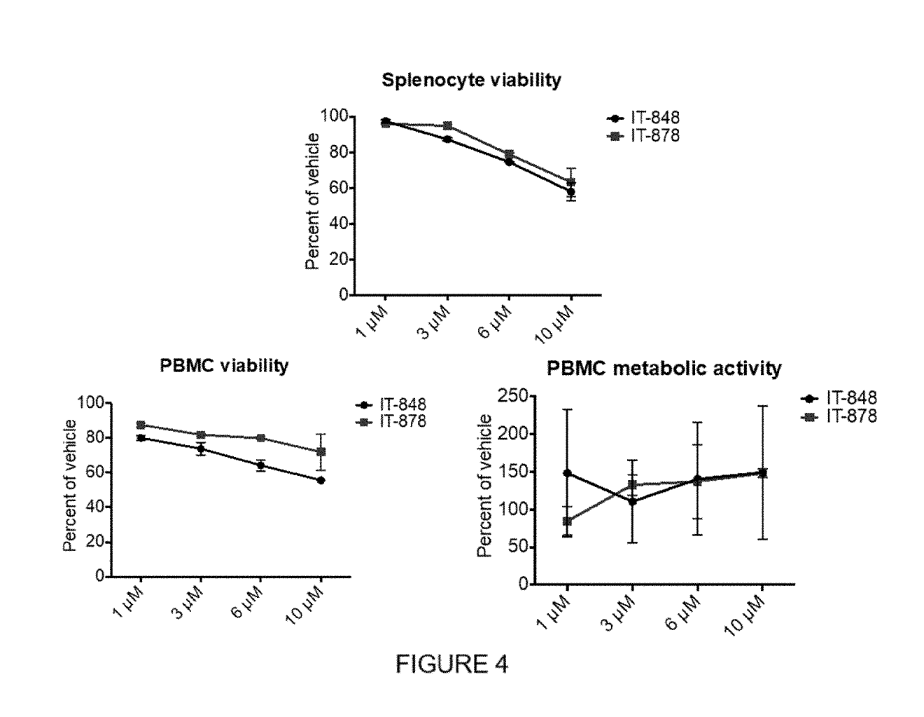

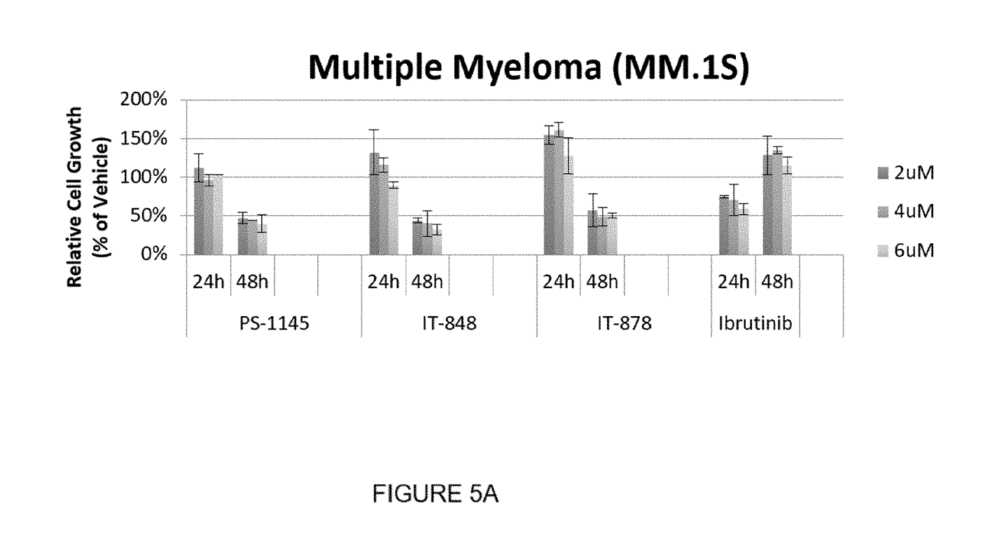

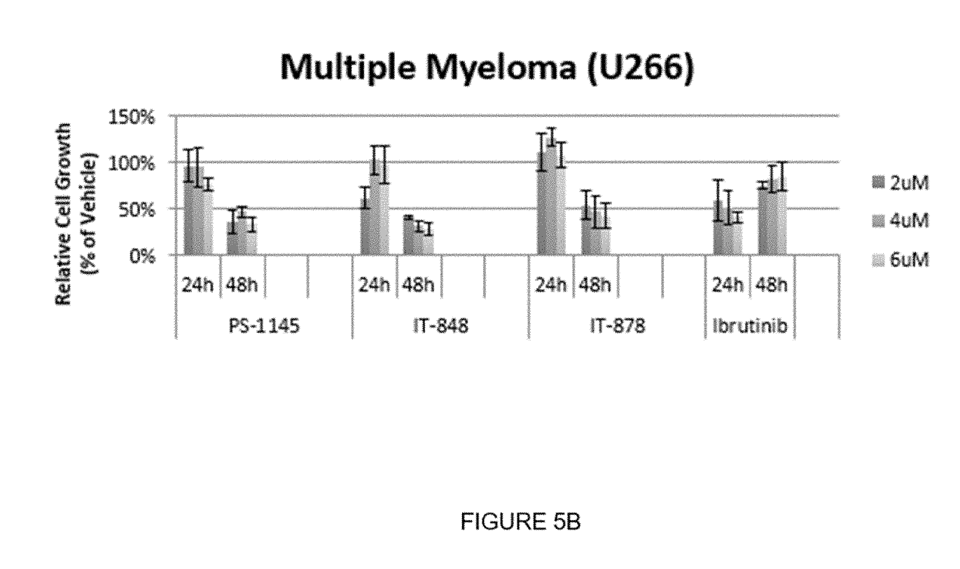

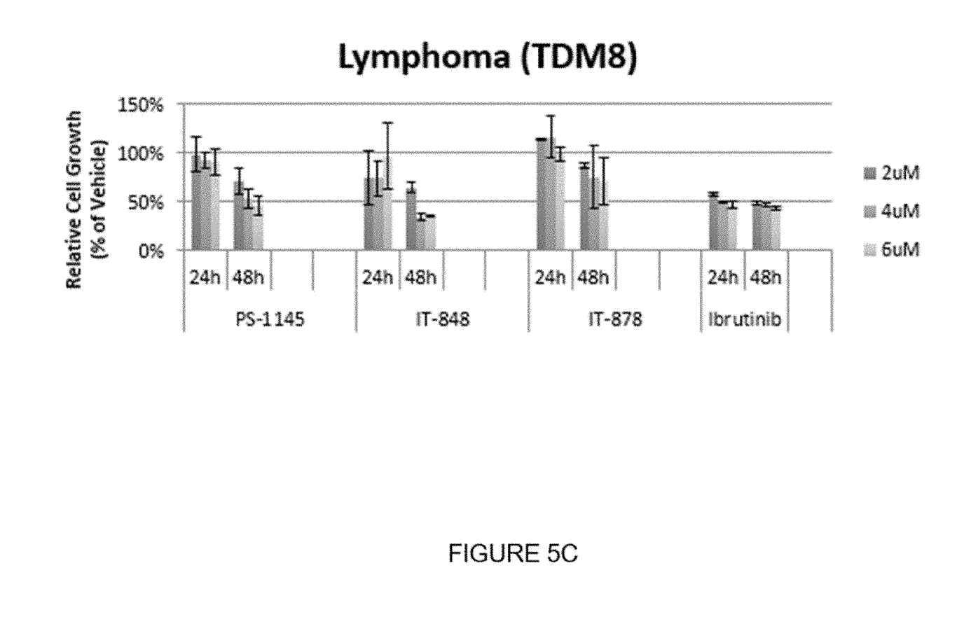

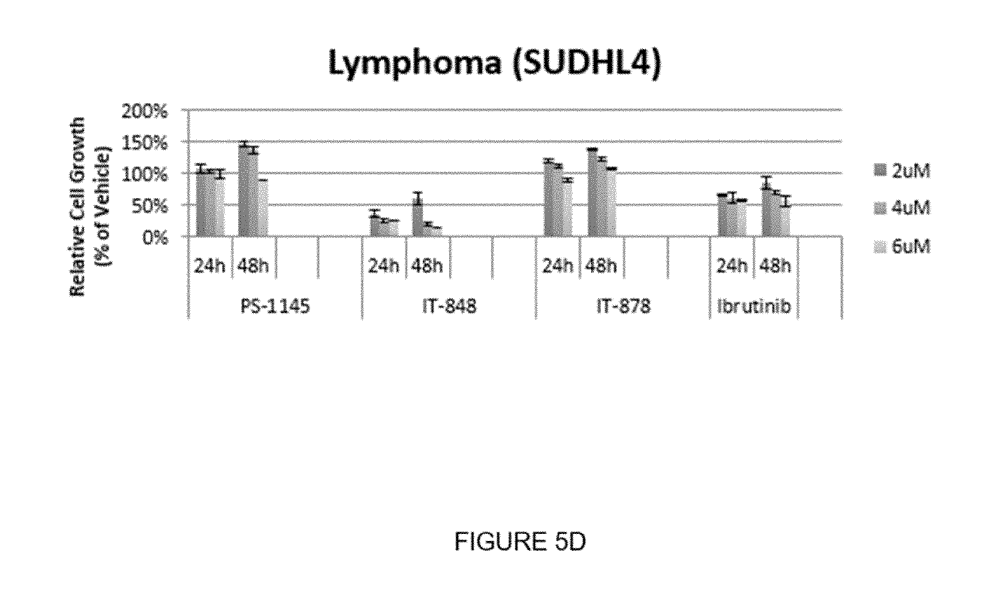

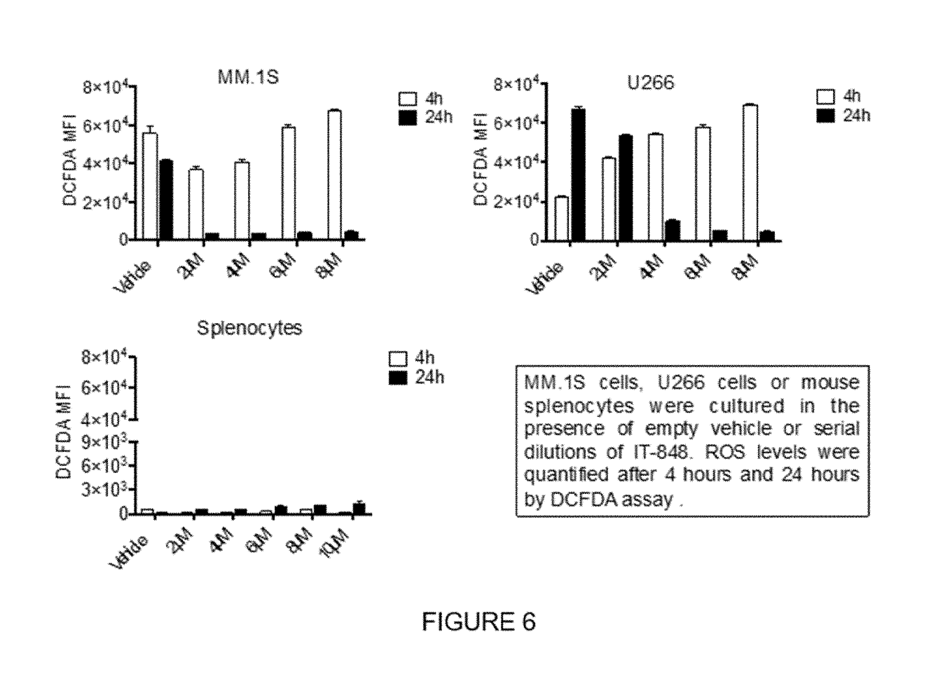

Next, specificity of IT-848 and IT-878 was assessed. Inhibition of transcriptional activity was specific to NF-kB but spared NFAT and Nrf2 (FIG. 3). In order to determine possible non-specific toxicities, healthy mouse splenocytes or human peripheral blood-derived mononuclear cells (PBMC) were cultured in the presence of serial dilutions of IT-848 and IT-878 (FIG. 4), revealing an acceptable level of compromise of viability and metabolic activity of healthy cells. We next analyzed anticancer properties of IT-848 in MTS assays of DLBCL and MM cells (FIG. 5). Two compounds (IT-848, IT-878) and known bioactive agents with NF-kB inhibitory activity (PS-1145, ibrutinib) were included as reference drugs. IT-848 displayed strong anti-lymphoma and anti-myeloma activity. Given that IT-848 has redox properties we evaluated its effect on the redox state of MM cells (FIG. 6). MM.1S and U266 cells had high levels of reactive oxygen species (ROS) at baseline and treatment with IT-848 resulted in unchanged or increased levels after four hours and decreased levels after 24 hours. Mouse splenocytes had 50 to 100-times lower ROS levels than MM cells and IT-848 treatment did not alter levels after 4 hours and led to a small increase after 24 hours. Modulation of the redox state of cancer cells that results in increased ROS levels after 4 hours followed by decreased ROS levels after 24 hours is associated with efficacy.

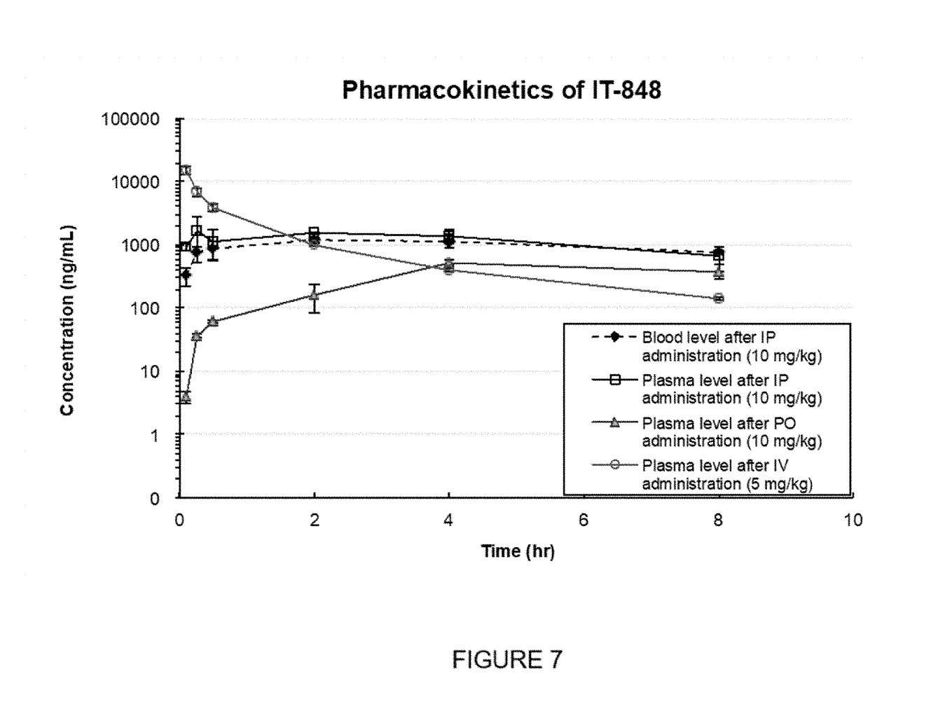

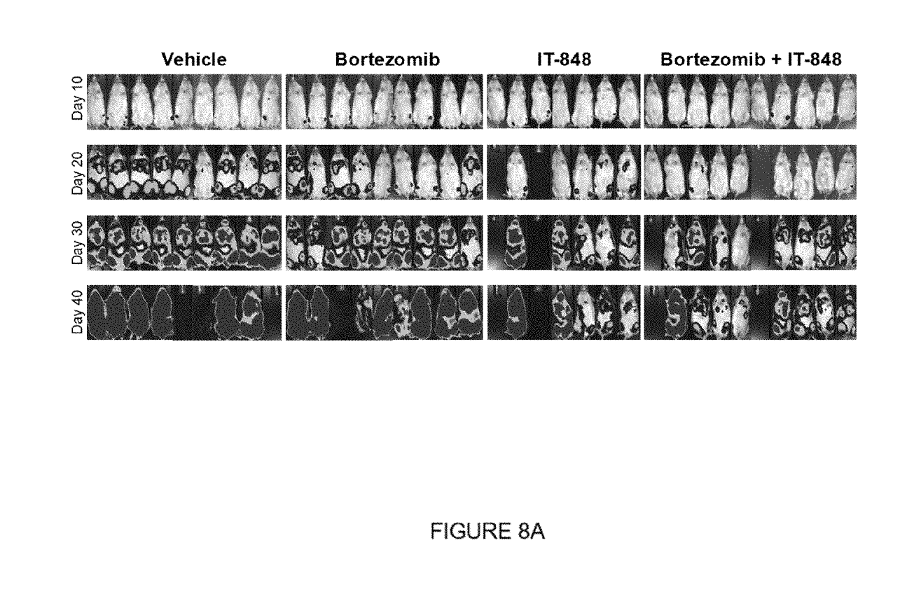

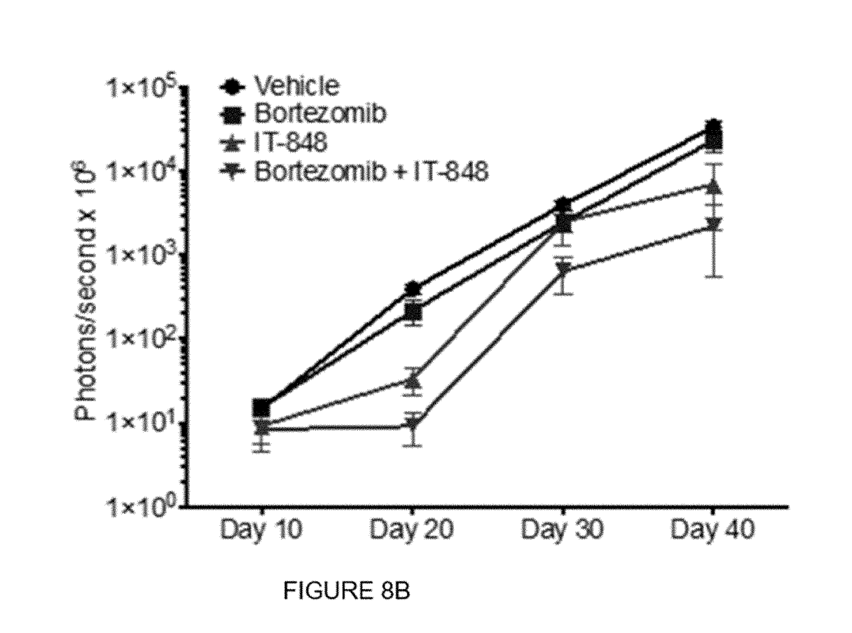

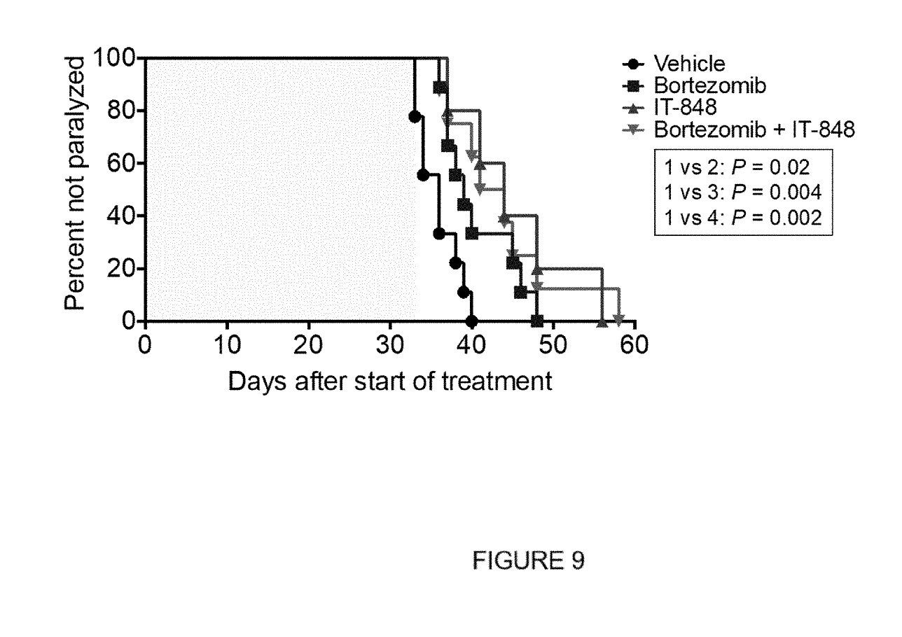

Next, we investigated pharmacokinetics (PK) of IT-848 by analyzing whole blood or plasma levels of IT-848 following oral (PO), intraperitoneal (IP), or intravenous (IV) administration (FIG. 7). Results indicate that clearance of IT-848 is slow, in particular after IP or oral administration, suggesting dosing every 24 hours or even longer dosing intervals should be sufficient to achieve the desired biological effect. Finally, in vivo efficacy studies were performed in a MM xenograft model. Immunodeficient mice received bioluminescent MM.1S cells intravenously and bone marrow engraftment was confirmed by in vivo bioluminescence imaging (BLI) after ten days. Mice were assigned to three treatment groups (IT-848 single agent, bortezomib single agent, combination of IT-848 and bortezomib) and a control group (empty vehicle). Onset of hind leg paralysis, which is the clinical endpoint of progressive disease in this model, was delayed in all three treatment groups (FIG. 9). In vivo BLI (FIG. 8A) including statistical analysis of longitudinal BLI data (FIG. 8B) revealed that both IT-848 and bortezomib were efficacious but combination treatment was best.

The following examples are provided to further illustrate the methods of the present disclosure. These examples are illustrative only and are not intended to limit the scope of the disclosure in any way.

### EXAMPLES

### Example 1: General Synthetic Scheme

The NF-κB inhibitors of the present disclosure can be synthesized by any of the suitable methods known in the art, or as further described below.

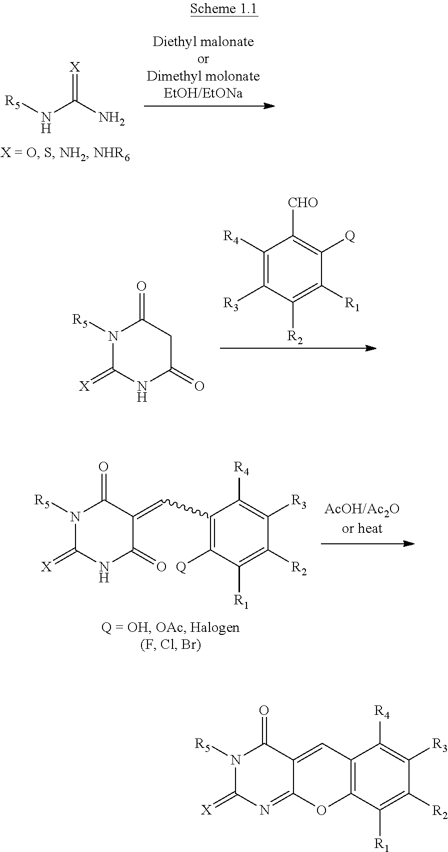

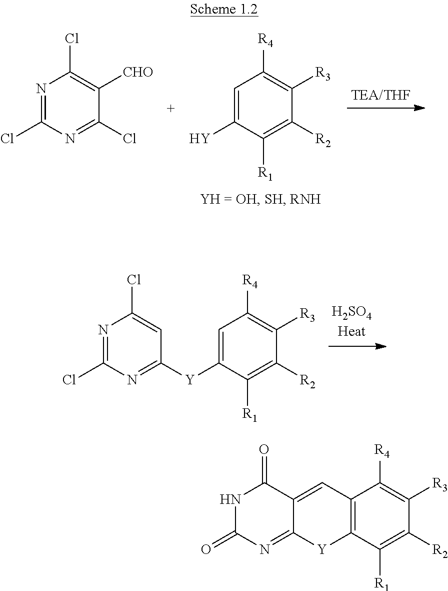

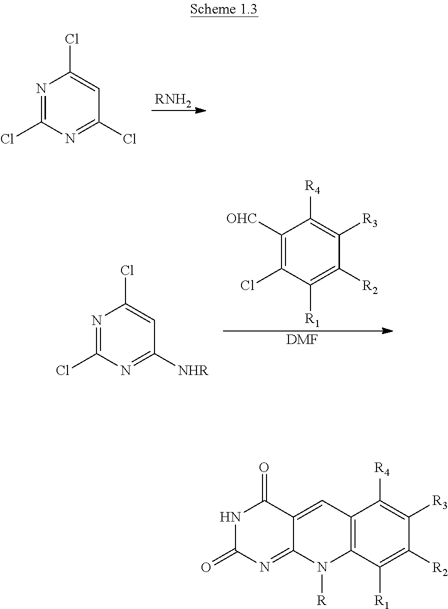

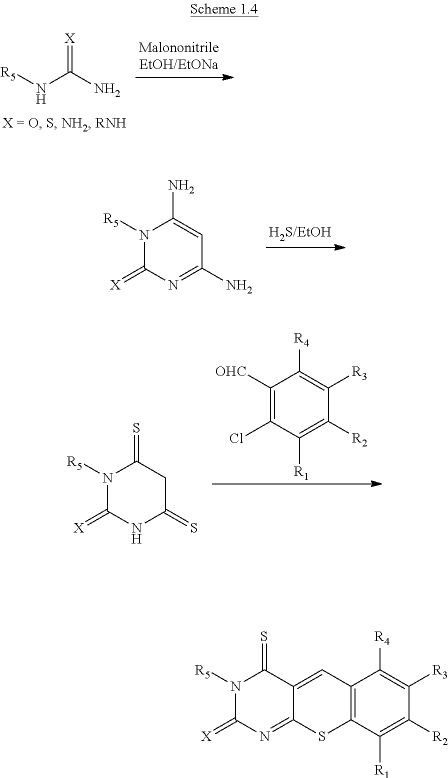

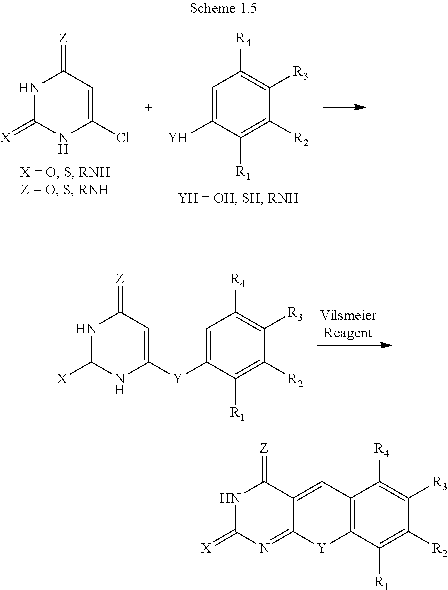

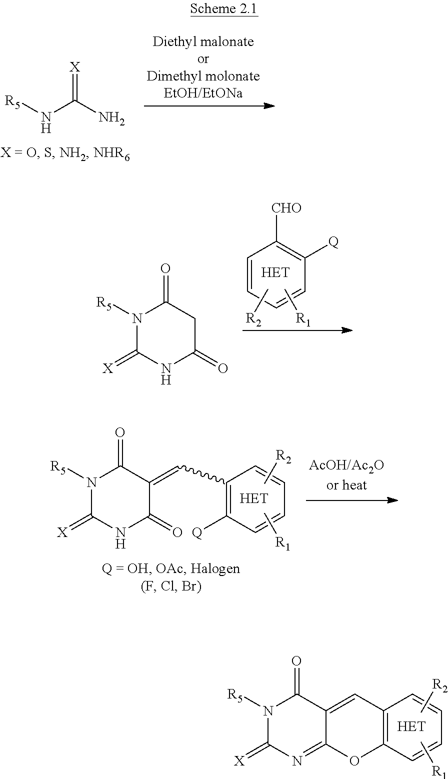

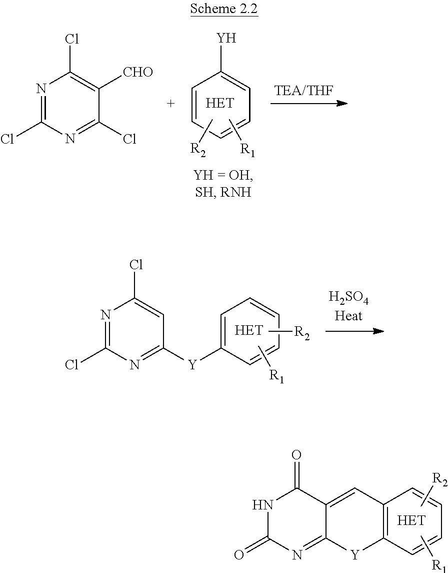

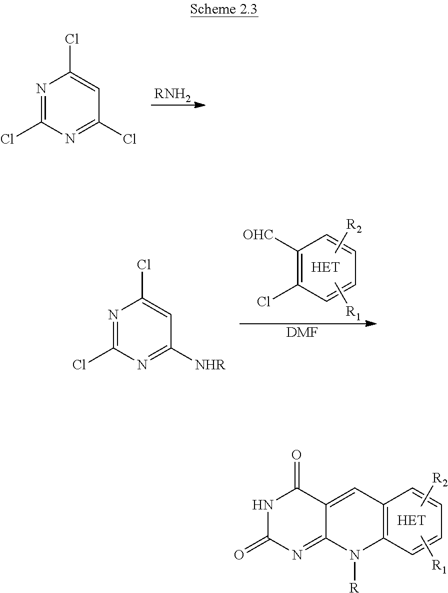

### Example 2: Compound-Specific Synthesis Protocols

### Preparation of IT-848

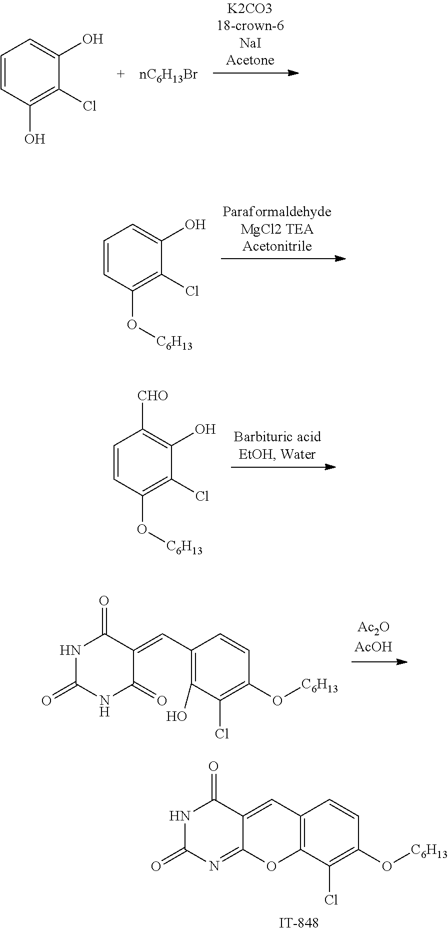

### Step 1: Preparation of 2-chloro-3-(hexyloxy)phenol

A 500 mL capacity 3-neck round bottom flash (reactor) was equipped with a magnetic stirrer, a reflux condenser and a thermometer. 18-crown-6 (3.17 g, 12 mmol), NaI (26.98 g, 180 mmol), K2CO3 (33.17 g, 240 mmol), 2-chlororesorcinol (29.91 g, 200 mmol), acetone (116 mL) and 1-bromohexane (29.17 g, 180 mmol) were successively added into the reactor in that order. The mixture was heated with oil bath. Temperature of oil bath was set at 72° C. Temperature inside the reactor reached 60° C. at steady state of the reaction. The mixture became more and more thick. An addition 29 mL of acetone was added into the mixture 5 hours after the reaction started. The reaction was stopped after 9 hours and cooled to room temperature. Once the reaction mixture reached room temperature, 150 mL of DI water and 150 mL of ethyl acetate were added into the reactor, consecutively. The organic layer was separated, washed with aq. NaOH, 1M, then with aq. NaOH, 0.5M, finally with brine. The organic layer (past brine washing) was filtered. The filtrate was concentrated by Rotovap to remove the maximum of ethyl acetate and to obtain a solid paste residue. To the solid paste residue were added 80 mL of hexanes and 50 mL of NaOH, 5M. The hexanes phase was separated and washed again with 50 mL of NaOH, 1M. The hexanes phase was dried over MgSO4, and concentrated to dryness to yield by-product, 2-chloro-1,3-bis(hexyloxy)benzene (13.35 g, 42.8 mmol, 12.4%). Combined aqueous phase was acidified with HCl, aq. 12M to pH=3, then extracted with 70 mL of ethyl acetate. The ethyl acetate phase was dried over MgSO4 and concentrated to dryness to obtain desired product, 2-chloro-3-(hexyloxy)phenol (20.88 g, 91.6 mmol, 45.8%).

### Step 2: Preparation of 3-chloro-4-(hexyloxy)-2-hydroxybenzaldehyde

A 250 mL capacity 3-neck round bottom flash (reactor) was equipped with a magnetic stirrer, a Claisen connector, a reflux condenser and a thermometer. 2-chloro-3-(hexyloxy)phenol (20.58 g, 90 mmol), MgCl2 (10.28 g, 108 mmol) and paraformaldehyde (6.75 g, 225 mmol) were charged into the reactor in that order. The reactor was placed under nitrogen. Acetonitrile (82 mL) and triethylamine (16.28 mL, 117 mmol) was loaded into the reactor in that order. The reaction was exothermic. The internal reaction temperature reached 49° C. The reactor was heated with oil bath to reach internal temperature of 60° C. The reaction mixture turned from light brown to yellow. The reaction mixture was heated for a total of 3 hours. The mixture was cooled to room temperature. The reaction mixture was transferred to a 500 mL Erlenmeyer. 90 mL of HCl aq. 1M was carefully and slowly added (exothermic) to the Erlenmeyer followed by an additional 300 mL of DI water and 300 mL of ethyl acetate. The mixture was stirred for 1 hour. The organic later was separated from the aqueous layer, washed with HCl, aq. 1M, and then brine. The organic layer was dried over MgSO4. The organic layer was concentrated to the maximum. The residue was sit overnight to allow desired product to crystallize. Crystal was filtered to yield desired product, 3-chloro-4-(hexyloxy)-2-hydroxybenzaldehyde (7.82 g, 30.4 mmol, 33.8%). Mother liquor was chromatographed to yield an additional desired product, (7.21 g, 28.1 mmol, 31.2%). Total yield is 65%.

### Step 3: Preparation of 5-(3-chloro-4-(hexyloxy)-2-hydroxybenzylidene)pyrimidine-2,4,6(1H,3H,5H)-trione

A 500 mL capacity 3-neck round bottom flash (reactor) was equipped with a magnetic stirrer and a thermometer. 3-chloro-4-(hexyloxy)-2-hydroxybenzaldehyde (6.42 g, 25 mmol) and EtOH (100 mL) was loaded into the reactor. The mixture was heated to allow 3-chloro-4-(hexyloxy)-2-hydroxybenzaldehyde to dissolve in ethanol. The mixture was allowed to cool to room temperature. Solid barbituric acid (3.52 g, 27.5 mmol) was added to the mixture. The mixture turned yellow and upon addition of barbituric acid. Following the addition of barbituric acid, 100 mL of water was added to the reaction mixture. The reaction mixture was stirred at room temperature for 36 hours. Yellow solid was filtered and vacuum dried to yield desired product (9.12 g, 24.8 mmol, 99%).

### Alternative Way to Prepare 5-(3-chloro-4-(hexyloxy)-2-hydroxybenzylidene)pyrimidine-2,4,6(1H,3H,5H)-trione

A 500 mL capacity 3-neck round bottom flash (reactor) was equipped with a magnetic stirrer and a thermometer. 3-chloro-4-(hexyloxy)-2-hydroxybenzaldehyde (8.98 g, 35 mmol) and iPrOH (55 mL) was loaded into the reactor. The mixture was heated to allow 3-chloro-4-(hexyloxy)-2-hydroxybenzaldehyde to dissolve in ethanol. The mixture was allowed to cool to room temperature. Solid barbituric acid (4.93 g, 38.5 mmol) was added to the mixture. The mixture turned yellow and upon addition of barbituric acid. Following the addition of barbituric acid, 55 mL of water was added to the reaction mixture. The reaction mixture was stirred at room temperature for 26 hours. 150 mL of water was added to the reaction mixture. The mixture was stirred for 30 minutes following the addition of water. Yellow solid was filtered and vacuum dried to yield desired product (12.70 g, 34.6 mmol, 99%).

### Step 4: Preparation of 9-chloro-8-(hexyloxy)-2H-chromeno[2,3-d]pyrimidine-2,4(3H)-dione, IT-848

A 250 mL capacity 3-neck round bottom flash (reactor) was equipped with a magnetic stirrer, a reflux condenser and a thermometer. The reactor was placed under nitrogen. 5-(3-chloro-4-(hexyloxy)-2-hydroxybenzylidene)pyrimidine-2,4,6(1H,3H,5H)-trione (20.17 g, 55 mmol), Acetic acid (132.11 g, 2.2 mol) and Acetic anhydride (22.46 g, 0.22 mol) were loaded into the reactor in that order. The reactor was heated with an oil bath set tween 80° C. to 100° C. to maintain slow reflux condensation. Internal temperature of the reaction mixture reached 78° C. The reaction was completed after 5 hours. Acetic acid and acetic anhydride was partially removed from the reaction mixture by Rotovap until the residue became a thick paste. The residue was re-suspended in a mixture of iPrOH (20 mL) and acetic acid (20 mL). Yellow solid was filtered. The cake was rinsed with a mixture of iPrOH (5 mL) and acetic acid (5 mL) and then with warm DI water. The yellow cake was vacuum dried to yield desired product, IT-848 (17.20 g, 49 mmol, 90%).

### Preparation of IT-878

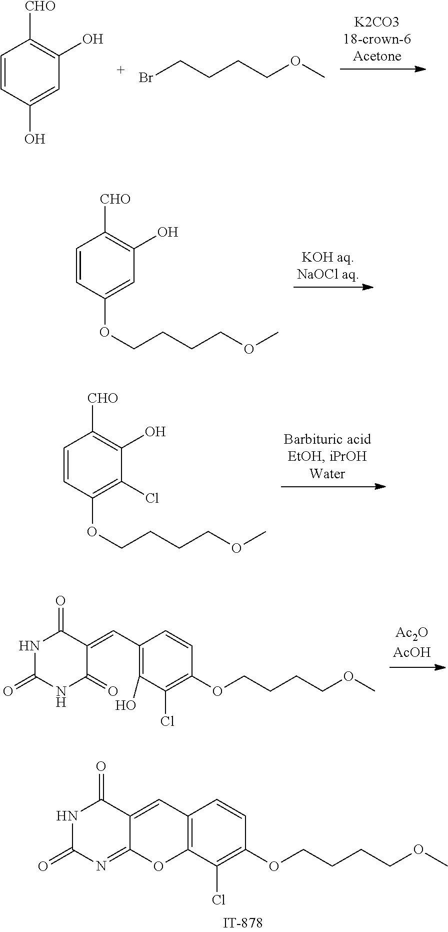

### Step 1: Preparation of 2-hydroxy-4-(4-methoxybutoxy)benzaldehyde

A 250 mL capacity 3-neck round bottom flash (reactor) was equipped with a magnetic stirrer, a reflux condenser and a thermometer. 18-crown-6 (2.64 g, 10 mmol), K2CO3 (20.01 g, 145 mmol), 2,4-dihydroxybenzaldehide (17.96 g, 130 mmol), acetone (130 mL) and 1-bromo-4-methoxybutane (24.96 g, 149.5 mmol) were successively added into the reactor in that order. The reactor was heated with oil bath to reflux for 15 hours. The reaction mixture was cooled down to room temperature. Acetone was evaporated from the reaction mixture by Rotovap. 150 mL of Hexanes was added to resuspend the residue obtain after acetone removal. Hexanes phase that contains dialkylated and monoalkylated products but not starting material, was separated by filtration. Solid obtained after filtration was dissolved with NaOH aq. 1M (150 mL). The mixture was extracted with hexanes (100 mL). Hexanes phase from filtration and from extracting were combined. The combined hexanes phase was washed with NaOH, aq. 1M (20 mL) to migrate monoalkylated desired product and starting material to aqueous layer and keep dialkylated product in organic layer. Combined aqueous layer was acidified with HCl, 50% to pH=8 and extracted with ethyl acetate. Ethyl acetate phase was dried over MgSO4, concentrated and chromatographed to yield 2-hydroxy-4-(4-methoxybutoxy)benzaldehyde (14.14 g, 63.1 mmol, 48.5%).

### Step 2: Preparation of 3-chloro-2-hydroxy-4-(4-methoxybutoxy)benzaldehyde

A 250 mL capacity 3-neck round bottom flash (reactor) was equipped with a magnetic stirrer and a thermometer. 2-hydroxy-4-(4-methoxybutoxy)benzaldehyde (13.4 g, 60 mmol) and DI water (20 mL) were added into the reactor. The reactor was cooled to with ice-bath. A solution of KOH aq. 45% (w/w) was slowly added to the mixture. The solution turned yellow. A aqueous solution of NaOCl, 8.25% (w/w) (108.4 g, 120 mmol) was added to the reactor over a period of 15 minutes. The reaction mixture was allowed to warm to room temperature and still for 2 hours. The starting material was totally consumed. The reaction mixture was acidified with HCl aq. 50% (v/v) to pH=1. The mixture was extracted with ethyl acetate (150 mL) twice. Combined organic layer was dried over MgSO4, concentrated and chromatographed to yield desired product, 3-chloro-2-hydroxy-4-(4-methoxybutoxy)benzaldehyde (8.25 g, 32 mmol, 53%).

### Step 3: Preparation of 5-(3-chloro-2-hydroxy-4-(4-methoxybutoxy)benzylidene)pyrimidine-2,4,6(1H,3H,5H)-trione

A 500 mL capacity 3-neck round bottom flash (reactor) was equipped with a magnetic stirrer and a thermometer. In the reactor, barbituric acid (4.51 g, 35.2 mmol) was suspended in DI water (100 mL). A solution of 3-chloro-2-hydroxy-4-(4-methoxybutoxy)benzaldehyde (8.25 g, 32 mmol) in a mixture of EtOH (50 mL) and iPrOH (50 mL) was added to the reactor. The reaction mixture was stirred for 7 hours. 150 mL was added to the reaction mixture. Yellow solid was filtered. LCMS indicated that the yellow solid contain 94% (area) of desired product and 6% (area) of 3-chloro-2-hydroxy-4-(4-methoxybutoxy)benzaldehyde. The yellow solid was resuspended in methanol at room temperature and then filtered to yield desired product, 5-(3-chloro-2-hydroxy-4-(4-methoxybutoxy)benzylidene)pyrimidine-2,4,6(1H,3H,5H)-trione (9.81 g, 26.6 mmol, 83%).

### Step 4: Preparation of 9-chloro-8-(4-methoxybutoxy)-2H-chromeno[2,3-d]pyrimidine-2,4(3H)-dione, IT-878

In a scintillation vial were added 5-(3-chloro-2-hydroxy-4-(4-methoxybutoxy)benzylidene)pyrimidine-2,4,6(1H,3H,5H)-trione (1.84 g, 5 mmol), acetic anhydride (1.53 g, 15 mmol) and acetic acid (12 g, 200 mmol). The vial was sealed and heated for 54 hours. The reaction was cooled to room temperature and sit overnight. The yellow solid was filtered. The yellow cake was washed with abundant warm water and vacuum dried to yield IT-878 (1.43 g, 4 mmol, 80%).

Jurkat/GFP/NF-κB transcriptional reporter cells were stimulated with TNF-α, Jurkat/Luciferase/NFAT transcriptional reporter cells were stimulated with PMA/ionomycin, and HepG2/Luciferase/Nrf2-Antioxidant response element (ARE) transcriptional reporter cells were treated with tert-butylhydroquinone. The respective cell lines were incubated for 12 hours in the presence of empty vehicle or 1, 3, 6 and 10 μM of IT-848 or IT-878. NF-κB transcriptional activity was analyzed by flow cytometry, NFAT and Nrf2 transcriptional activities were analyzed by luminescence measurement. The results are shown in FIG. 3. Mean and SEM of relative fluorescent or luminescent intensities are presented.

### Example 4: Mice, Cell Lines and Primary Cells

### Cell Lines and Primary Cells

Human peripheral blood mononuclear cells (PBMC) were purified from donor blood according to accepted protocols. Mouse splenocytes were purified from mouse spleen using methods known to those skilled in the art.

The human DLBCL-derived cell lines SU-DHL4 and U2932 were obtained from the German Collection of Microorganisms and Cell Cultures, Department of Human and Animal Cell Cultures (Braunschweig, Germany); MM.1S and U266 cells were obtained from ATCC; HBL1 cells were kindly provided by Dr. R. E. Davis (Houston, Tex.) and authenticated by the MD Anderson Characterized Cell Lines Core Facility. MM.1S cells were retrovirally transduced to express a fusion protein consisting of enhanced green fluorescent protein and firefly luciferase. Cell lines were cultured in RPMI 1640 medium supplemented with 10 to 20% fetal bovine serum, 1% L-glutamine, and penicillin-streptomycin in a humid environment of 5% CO₂ at 37° C.

A NF-κB/Jurkat/GFP transcription reporter cell line was purchased from System Biosciences. NFAT/Jurkat/Luciferase transcriptional reporter cells were generated at Memorial Sloan-Kettering Cancer Center. HepG2/ARE/Luciferase Nrf2 transcriptional reporter cells were purchased from BPS biosciences.

Patient-derived lung cancer, ovarian cancer and breast cancer cells were purchased from Cell Biologics (Chicago Ill.).

### Mice

Female NOD/scid/IL2Rγ(null) (NSG) mice were purchased from the mouse Genetics Core Facility at MSKCC.

### Example 5: Assays

### Detection of Reactive Oxygen Species

Cells were treated in vitro with IT-848 (compound of Formula I), IT-878 (compound of Formula II) or vehicle solution. Reactive oxygen species (ROS) levels were analyzed by cell labelling with 2′, 7′-dichlorofluorescein diacetate (DCFDA) using a Cellular Reactive Oxygen Species Detection Assay Kit (Abcam, Cambridge, Mass.) ROS levels were analyzed by quantifying the fluorogenic marker DCFDA using flow cytometery (LSR II, BD Bioscience, San Jose, Calif.).

### Pharmacokinetics

Plasma or whole blood samples were analyzed at several time points after intraperitoneal, intravenous or oral administration of 5-10 mg/kg of IT-848. Samples were analyzed by liquid chromatography-tandem mass spectrometry (LC-MS/MS). The results are shown in FIG. 7. IT-848 is cleared slowly after systemic administration.

### In Vitro Anti-Tumor Assay

TDM8, Ly19, L363, SUDHL4, MM.1S or U266 cells were independently incubated for up to 48 hours in the presence of empty vehicle, IT-compound (2, 4, 6 μM), PS-1145 (2, 4 and 6 μM), or ibrutinib (2, 4 and 6 μM). Cell growth was measured indirectly after 24 and 48 hours by CellTiter 96 Aqueous Non-Radioactive Cell Proliferation Assay (Promega, Madison, Wis.) per manufacturer's instructions. Briefly, tetrazolium compound [-(4,5-dimethylthiazol-2-yl)-5-(3-carboxymethoxyphenyl)-2-(4-sulfophenyl)-2H-tetrazolium, inner salt; MTS] was added to the culture media and the conversion of MTS into formazon was measured by the amount of 490 nm absorbance. The number of viable cells correlates with absorbance at 490 nm. The results are shown in FIGS. 5A-D. Mean and SEM of relative cell growth are presented.

### Example 6: Compounds' Inhibition of Healthy Cells

Mouse splenocytes and human peripheral blood mononuclear cells (PBMC) were incubated for 24 hours in the presence of empty vehicle or 1, 3, 6 and 10 μM of IT-848 or IT-878. Viability (percentage of DAPI-negative cells) was analyzed by flow cytometry and metabolic activity was analyzed by MTS assay. Mean and SEM of relative viability or metabolic activity are presented.

### Example 7: In Vivo Efficacy in Xenograft Mouse Model

### Mice

Female NOD/scid/IL2Rγ(null) (NSG) mice were purchased from the Mouse Genetics Core Facility at MSKCC.

### In Vivo Bioluminescent Signal Intensity (BLI)

In tumor xenograft experiments the bioluminescent signal intensity (BLI) of tumor bearing mice was determined weekly. As shown in FIG. 8A, pseudocolor images showing the whole-body distribution of bioluminescent signal intensity was superimposed on grayscale photographs and total flux (photons s⁻¹) for individual mice was determined. The cause of death (tumor versus toxicity) was determined based on the presence of hind leg paralysis. Death in the absence of hind leg paralysis was attributed to toxicity. Animals with significantly impaired mobility due to paralysis were euthanized. As shown in FIG. 9, IT-848 delays the onset of paralysis in a xenograft model of multiple myeloma.

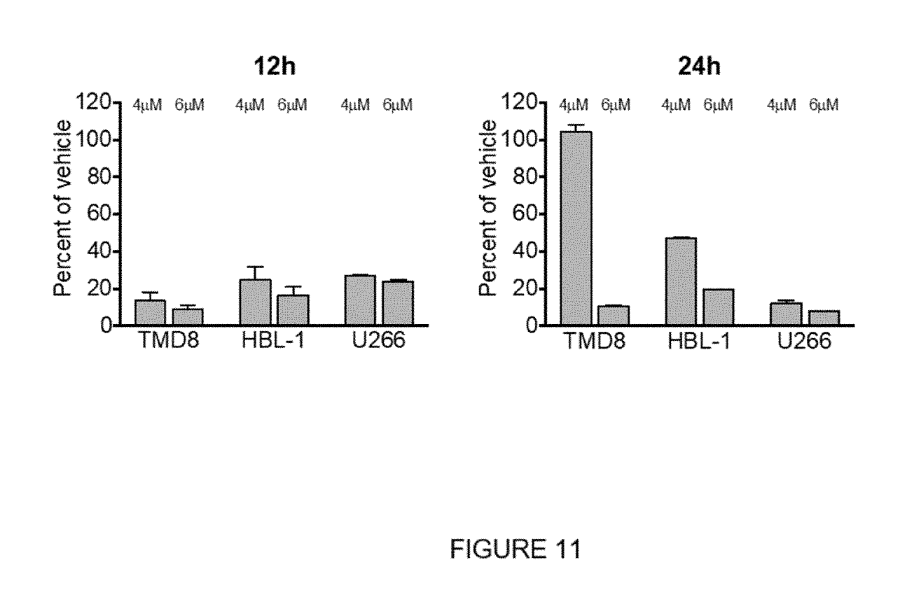

Using Western Blot and transcriptional reporter assays we previously demonstrated that IT-848 and IT-878 were potent NF-kB inhibitors, and based on additional in vitro and in vivo efficacy studies IT-848 (formula I) was selected as a drug candidate. To confirm the potency of IT-848 expression of surrogate markers of NF-kB activity in B cell malignancies (the NF-kB target genes IL-6 and IL-10) in diffuse large B cell lymphoma (DLBCL) and Multiple Myeloma (MM) cell lines treated with IT-848 was analyzed (FIGS. 10 and 11). Secretion of both cytokines was decreased after 12 and 24 hours of treatment in TMD8 cells (DLBCL), HBL1 cells (DLBCL), and U266 cells (MM).

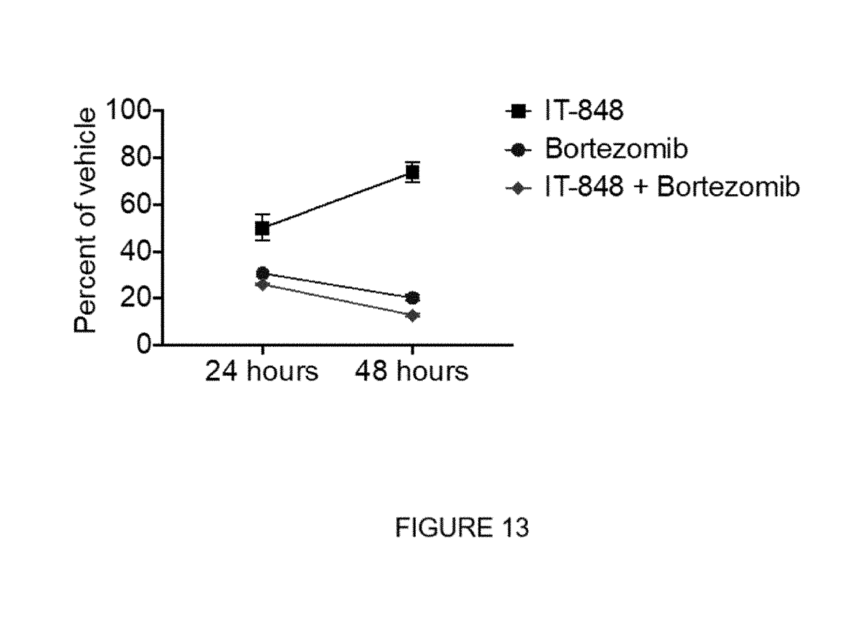

Efficacy studies of IT-848 combined with drugs that are currently used clinically to treat B cell malignancies were also performed. MTS assays in TMD8 and HBL1 cells revealed that IT-848 can enhance the efficacy of bortezomib or panobinostat (FIGS. 12 and 13). Moreover, IT-848 was efficacious in a xenograft model of MM (FIGS. 14, 15A and 15B) and enhanced the efficacy of bortezomib (FIGS. 15A and 15B). Immunodeficient mice received bioluminescent MM.1S cells intravenously and bone marrow engraftment was confirmed by in vivo bioluminescence imaging (BLI) after ten days. Mice were assigned to three treatment groups (IT-848 single agent, bortezomib single agent, combination of IT-848 and bortezomib) and a control group (empty vehicle IP). Onset of hind leg paralysis, which is the clinical endpoint of progressive disease in this model, was delayed in all three treatment groups (FIG. 9). In vivo BLI (FIG. 8A) including statistical analysis of longitudinal BLI data (FIG. 8B) revealed that both IT-848 and bortezomib were efficacious but combination treatment was best.

### REFERENCES

1. Gorrini, C., Harris, I. S. & Mak, T. W. Modulation of oxidative stress as an anticancer strategy. Nature reviews. Drug discovery 12, 931-947 (2013).
2. Trachootham, D., Alexandre, J. & Huang, P. Targeting cancer cells by ROS-mediated mechanisms: a radical therapeutic approach? Nature reviews. Drug discovery 8, 579-591 (2009).
3. Hass, C., Belz, K. & Fulda, S. Sensitization of acute lymphoblastic leukemia cells for LCL161-induced cell death by targeting redox homeostasis. Biochemical pharmacology (2016).
4. Irwin, M. E., Rivera-Del Valle, N. & Chandra, J. Redox control of leukemia: from molecular mechanisms to therapeutic opportunities. Antioxidants & redox signaling 18, 1349-1383 (2013).
5. Schoeneberger, H., Belz, K., Schenk, B. & Fulda, S. Impairment of antioxidant defense via glutathione depletion sensitizes acute lymphoblastic leukemia cells for Smac mimetic-induced cell death. Oncogene 34, 4032-4043 (2015).
6. Hsieh, M. Y. & Van Etten, R. A. IKK-dependent activation of NF-kappaB contributes to myeloid and lymphoid leukemogenesis by BCR-ABL1. Blood 123, 2401-2411 (2014).
7. Kordes, U., Krappmann, D., Heissmeyer, V., Ludwig, W. D. & Scheidereit, C. Transcription factor NF-kappaB is constitutively activated in acute lymphoblastic leukemia cells. Leukemia 14, 399-402 (2000).
8. Lavorgna, A. & Harhaj, E. W. EBV LMP1: New and shared pathways to NF-kappaB activation. Proceedings of the National Academy of Sciences of the United States of America 109, 2188-2189 (2012).
9. Kaye, K. M., Izumi, K. M. & Kieff, E. Epstein-Barr virus latent membrane protein 1 is essential for B-lymphocyte growth transformation. Proceedings of the National Academy of Sciences of the United States of America 90, 9150-9154 (1993).
10. Shair, K. H., et al. EBV latent membrane protein 1 activates Akt, NFkappaB, and Stat3 in B cell lymphomas. PLoS pathogens 3, e166 (2007).
11. Gasparini, C., Celeghini, C., Monasta, L. & Zauli, G. NF-kappaB pathways in hematological malignancies. Cellular and molecular life sciences: CMLS 71, 2083-2102 (2014).
12. Shumilla, J. A., Wetterhahn, K. E. & Barchowsky, A. Inhibition of NF-kappa B binding to DNA by chromium, cadmium, mercury, zinc, and arsenite in vitro: evidence of a thiol mechanism. Archives of biochemistry and biophysics 349, 356-362 (1998).
13. Morgan, M. J. & Liu, Z. G. Crosstalk of reactive oxygen species and NF-kappaB signaling. Cell research 21, 103-115 (2011).
14. Mihm, S., Galter, D. & Droge, W. Modulation of transcription factor NF kappa B activity by intracellular glutathione levels and by variations of the extracellular cysteine supply. FASEB journal: official publication of the Federation of American Societies for Experimental Biology 9, 246-252 (1995).
15. Shono, Y., et al. A small-molecule c-Rel inhibitor reduces alloactivation of T cells without compromising antitumor activity. Cancer discovery 4, 578-591 (2014).
16. Shono, Y., et al. Characterization of a c-Rel inhibitor that mediates anticancer properties in hematologic malignancies by blocking NF-kappaB-controlled oxidative stress responses. Cancer research 76, 377-389 (2016).
17. Na, I. K., et al. Concurrent visualization of trafficking, expansion, and activation of T lymphocytes and T-cell precursors in vivo. Blood 116, e18-e25 (2010).

We claim:

1. A method for inhibiting growth and proliferation of cells selected from the group consisting of lymphoma cells and myeloma cells comprising contacting the cells with a compound selected from the group consisting of: 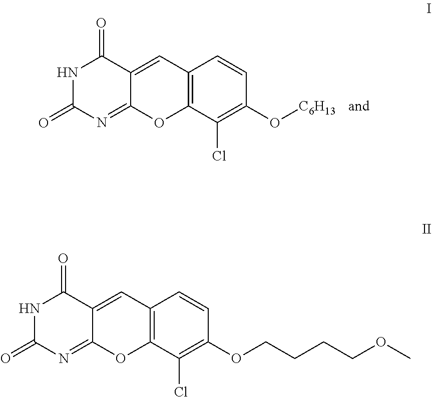
and crystalline forms, hydrates, or pharmaceutically acceptable salts thereof.

2. The method of claim 1, wherein the lymphoma cells are diffuse large B-cell lymphoma (DLBCL) cells.

3. The method of claim 1, wherein the myeloma cells are multiple myeloma (MM) cells.

4. The method of claim 1, 2 or 3, wherein the cells are contacted with a compound of formula I: 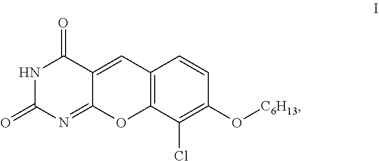
or a crystalline form, hydrate or pharmaceutically acceptable salt thereof.

5. The method of claim 1, 2 or 3, wherein the cells are contacted with a compound of formula II: 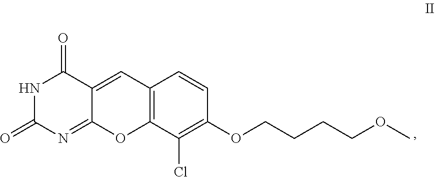 [SMILES: COCCCCOc1ccc2cc3c(=O)[nH]c(=O)nc-3oc2c1Cl]
a crystalline form, hydrate or pharmaceutically acceptable salt thereof.

6. A method for treating a subject with myeloma, comprising administering to the subject a therapeutically effective amount of a compound selected from the group consisting of: 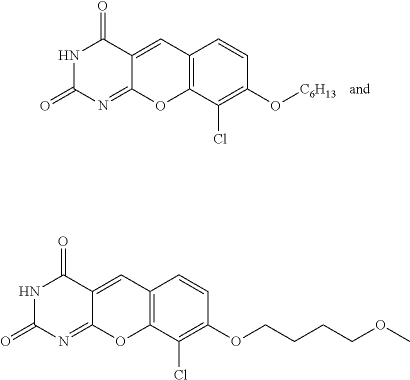
and crystalline forms, hydrates, or pharmaceutically acceptable salts thereof.

7. The method of claim 6, wherein the myeloma is multiple myeloma.

8. The method of claim 6 or 7, wherein a therapeutically effective amount of a compound of formula I 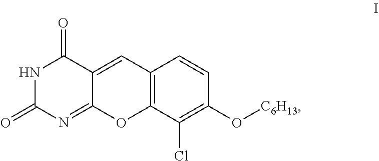
a crystalline form, hydrate or pharmaceutically acceptable salt thereof is administered to the subject.

9. The method of claim 6 or 7, wherein a therapeutically effective amount of a compound of formula II 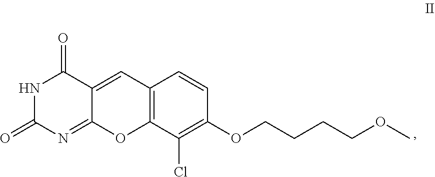 [SMILES: COCCCCOc1ccc2cc3c(=O)[nH]c(=O)nc-3oc2c1Cl]
a crystalline form, hydrate or pharmaceutically acceptable salt thereof is administered to the subject.

10. A method for treating a subject with lymphoma, comprising administering to the subject a therapeutically effective amount of a compound selected from the group consisting of: 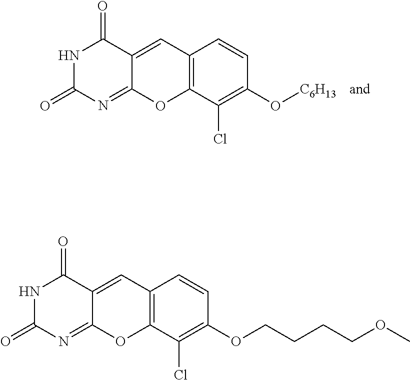
and crystalline forms, hydrates, or pharmaceutically acceptable salts thereof.

11. The method of claim 10, wherein the lymphoma is diffuse large B-cell lymphoma (DLBCL).

12. The method of claim 10 or 11, wherein a therapeutically effective amount of a compound of formula I 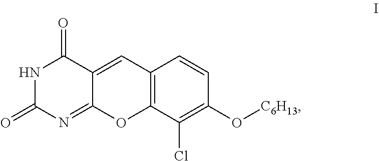
a crystalline form, hydrate or pharmaceutically acceptable salt thereof is administered to the subject.

13. The method of claim 10 or 11, wherein a therapeutically effective amount of a compound of formula II 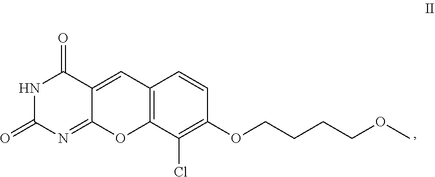 [SMILES: COCCCCOc1ccc2cc3c(=O)[nH]c(=O)nc-3oc2c1Cl]
a crystalline form, hydrate or pharmaceutically acceptable salt thereof is administered to the subject.

14. A compound capable of inhibiting NF-κB selected from the group consisting of 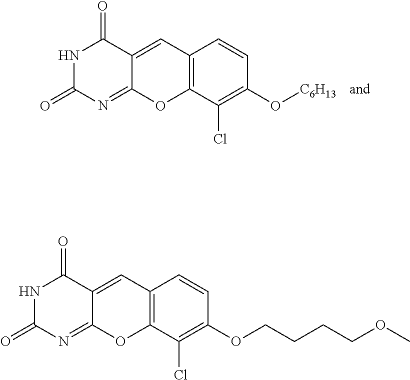
and crystalline forms, hydrates, or pharmaceutically acceptable salts thereof for use in the treatment of myeloma or lymphoma.

15. A pharmaceutical composition comprising a pharmaceutically acceptable carrier and a compound according to claim 15.

16. Use of the composition of claim 15 for the treatment of myeloma or lymphoma.

17. The use of claim 17, wherein the myeloma is multiple myeloma.

18. The use of claim 17, wherein the lymphoma is diffuse large B-cell lymphoma (DLBCL).

19. A pharmaceutical composition comprising a compound selected from the group consisting of: 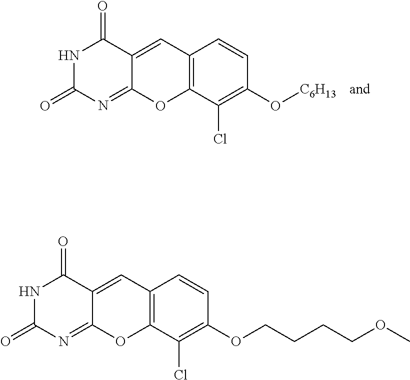
and crystalline forms, hydrates, or pharmaceutically acceptable salts thereof in combination with bortezomib or panobinostat.

20. A pharmaceutical composition of claim 19 comprising 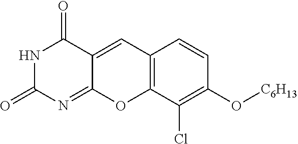
or a crystalline form, hydrate, or pharmaceutically acceptable salt thereof in combination with bortezomib or panobinostat.

21. A pharmaceutical composition of claim 19 comprising 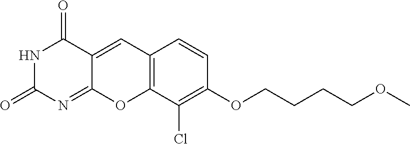 [SMILES: COCCCCOc1ccc2cc3c(=O)[nH]c(=O)nc-3oc2c1Cl]
or a crystalline form, hydrate, or pharmaceutically acceptable salt thereof in combination with bortezomib or panobinostat.

22. A method for treating a subject with lymphoma or myeloma, comprising administering to the subject a therapeutically effective amount of a compound selected from the group consisting of: 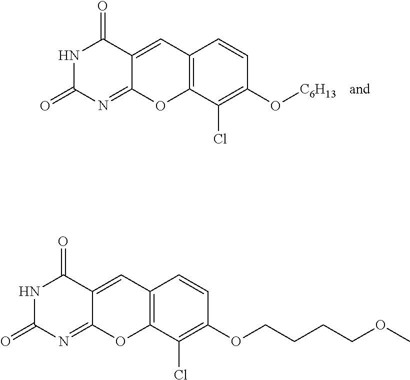
and crystalline forms, hydrates, or pharmaceutically acceptable salts thereof in combination with bortezomib or panobinostat.
